(19) United States
(12) Patent Application Publication
(10) Pub. No.: US 2012/0142621 A1
(43) Pub. Date: Jun. 7, 2012

(54) Purified Ethyl Ester Sophorolipid for the Treatment of Sepsis

(75) Inventors: George Falus, New York, NY (US); Maja Nowakowski, Port Washington, NY (US); Martin Bluth, Southfield, MI (US); John Aikens, Oak Brook, IL (US)

(73) Assignee: Biomedica Management Corporation, Brooklyn, NY (US)

(21) Appl. No.: 13/196,014

(22) Filed: Aug. 2, 2011

Related U.S. Application Data
(60) Provisional application No. 61/419,272, filed on Dec. 3, 2010.

(51) Int. Cl. A61K 31/7048; A61P 31/00; C12P 19/62

(52) U.S. Cl. 514/31; 435/76

(57) ABSTRACT

A microbial ethyl esther sophorolipid derivative with no acetylated groups produced by Candida species, for treating and preventing sepsis/septic shock. The method of producing sophorolipids is through microbial resting cells of Candida bombicola. The sophorolipids obtained from resting state cultures are isolated as a complex mixture of compounds and then decanted as a dense oil from the culture broth, subsequently washed to remove free fatty acids. Secondary chemical transformation via base catalyzed hydrolysis is used to reduce the 8 possible structural sophorolipid species to a single moiety, the 17-L-[(2′-O-b-D-glucopyranosyl-b-D-glucopyranosyl)-oxy]-cis-9-octadecenoate de-acetylated free acid. The compound acts primarily through decreasing inflammatory cytokines and eliciting other synergistic anti-inflammatory mechanisms by blocking TLR4-CD14 upstream of the inflammatory signaling cascade. The compound can be administered either intraperitoneally or intravenously at single or multiple doses of 5-30 mg/kg of weight in solvent media or in capped nanoparticles, preferably within 48 hours of sepsis inception.

### RELATED APPLICATIONS

Domestic priority is claimed from U.S. Provisional patent application No. 61/419,272 filed Dec. 3, 2010 and entitled Non-acetylated Ethyl Ester Sophorolipid for the Treatment of Sepsis, the entirety of which is hereby incorporated by reference herein.

### BACKGROUND OF THE INVENTION

1. Field of the Invention

The present invention relates for the production of ethyl esther sophorolipid derivative with no acetylated groups, which may be used to prevent and treat sepsis and septic shock. The sophorolipid is produced by a method involving reacting a compound of formula 17-L-[(2′-O-b-D-glucopyranosyl-b-D-glucopyranosyl)-oxy]-cis-9-octadecenoate de-acetylated free acid.

2. Description of Related Art

Severe sepsis is a complex clinical entity with mortality remaining unacceptably high—30 to 50 percent. There is great interest in identifying novel strategies to treat not only infections, but also the associated inflammatory responses. We postulate that sophorolipids are novel therapeutic candidates for the treatment of sepsis, and act primarily through decreasing inflammatory cytokines and eliciting other synergistic anti-inflammatory mechanisms. Microbial sophorolipids are extracellular glycolipids produced by Candida species when grown on mixtures of carbohydrates and fatty acids. Typically, sophorolipids consist of a dimeric sophorose connected by a glycosidic bond to the penultimate hydroxyl group of an 18-carbon fatty acid.

The sophorolipid produced and its structural analogues have been studied for its spermicidal and anti-I-fly activities. Sophorolipid di-acetate ethyl ester derivative is the most potent spermicidal and virucidal agent of the series of SLs studied. Its anti-viral activity against HIV and sperm-immobilizing activity against human semen are similar to those of commercial spermicide Nonoxynol-9.

Sophorolipids and their derivatives have also shown promise as surfactants, emulsifiers. Antimicrobials, anti-inflammatory and a source of specialty chemicals such as sophorose and hydroxylated fatty acids. There has been considerable interest in the physiological properties of sophorolipids, which have shown exciting potential in the treatment of a host of disorders. SLs have been reported to have caused differentiation and protein kinase C inhibition in the HL6O leukemia cell line.⁷ Additionally they are useful as immunomodulators for Parkinson's disease, Alzheimer's disease, psoriasis, AIDS treatment, as well as for antiviral immunostimulation.⁸ Consequently, there has been a great deal of interest in the synthesis of novel SL derivatives. To date, however, the primary strategy identified for the tailoring of SL structure has been during in vivo formation by the selective-feeding of lipophilic substrates. For example, changing the co-substrate from sunflower to canola oil resulted in a large increase (50-73%) of the lactonic portion of SLs.” Interestingly, using oleic acid (alone or with glucose) increased the fraction of non-acetylated I′.4″ sophorolipid lactonic. Unsaturated fatty acids such as oleic acid may be incorporated unchanged into sophorose lipids.

It is stated in U.S. Pat. Nos. 2,205,150 and 3,212,684 that a quantity of sophorolipids was produced by a fermentation process using a culture of Torulopsis bombicola, a strain presently classified as Candida bombicola. The prior art is also described in U.S. Pat. No. 3,445,337 and in Journal of the American Oil Chemistry Society, vol. 65, no. 9, September 1988, pp. 1460-1466. French patent application 2670798 also describes a process for the production of sophorolipids by fermentation with continuous supply or fed batch of esters of fatty acids or oils.

### Producing Microorganisms

In the early 1960s of the past century, Gorin et al (1961) were the first to describe an extracellular glycolipid synthesized by the yeast Torulopsis magnoliae. The structure of the hydroxy fatty acid sophoroside mixture was elucidated as a partially acetylated 2-O-β-d-glucopyranosyl-dl.-glucopyranose unit attached β-glycosidically to 17-t.-hydroxyoctadecanoic or 17-t-hydroxy-A9-octadecenoic acid (Tulloch et at 1962—Tulloch and Spencer 1968).

In the same year, Iulloch et al. also discovered a new sophorolipid from Candida bogrniensis with a similar structure but different in its hydroxy fatty acid moiety: the sophorose unit is linked to 13-hydroxydocosanoic acid. More recently several sophorolipid secreting yeast strains were identified.

Regarding the fact that the production of sophorolipids is not restricted to one single yeast species, but to a number of related microorganisms, it is not unlikely to presume that other species belonging or related to the Candida are also capable to synthesize some sort of sophorolipid. Nontheless, C bambicola ATCC 22214 is the strain preferred by most research groups active in the sophorolipid field because it can produce over 400 g/l sophorolipids and is used for commercial production and applications.

The building blocks for conventional sophorolipid synthesis are glucose and a fatty acid. Ideally, both can be provided in the production medium as such or, because free fatty acids can disturb the electron balance of the cells, sometimes fatty acid methyl or ethyl esters, or triglycerides are used.

The sophorolipids obtained after the action of glucosyltransferase II are as such detected in the sophorolipid mixture as the acidic, nonacetylated molecules. The majority of the sophorolipids are however further modified by both internal esterification (lactonization) and by acetylation of the carbohydrate bead.

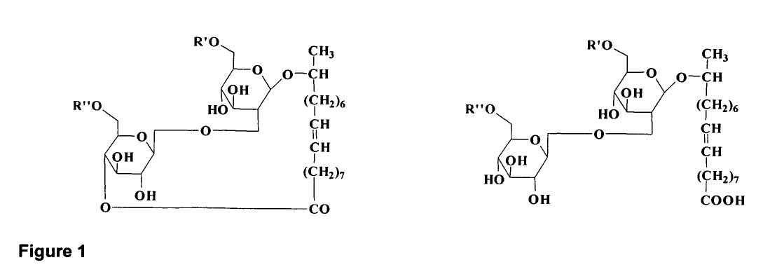

The sophorolipids obtained are considered as being a mixture of compounds representing the acid form and the lactone form as shown in FIG. 1.

In these formulas, R represents hydrogen or an acetyl group and R 2 hydrogen or an alkyl radical having 1 to 9 carbon atoms, when R is a saturated hydrocarbon radical with 7 to 16 carbon atoms, or R 2 represents hydrogen or a methyl group, when R is an unsaturated hydrocarbon radical with 13 to 17 carbon atoms.

Various homologues and the separation of one or the main forms (acid or lactone) e.g. have been described. This separation requires extractions by a specific solvent does not always give good results because the solubility of all the homologues in a solvent can differ significantly, which affects the quality of the products obtained.

Deacetylated esters or acids can be obtained by methanolysis reactions in the presence of an acid catalyst

Finally, it is known that the acetyl bonds in sophorolipids are chemically unstable and are very easily hydrolyzed by heating or prolonged storage close to neutrality or even at ambient temperature under slightly alkaline conditions, which leads to the obtaining of the completely deacetylated acid form. It is therefore extremely difficult by fermentation or chemistry to obtain a single product and a fortiori an acetylated product.

Moreover, in petroleum applications e.g. linked with the assisted recovery of petroleum, it is necessary to be able to create water-in-oil emulsions and therefore to be able to have emulsion products which are more hydrophobic than hydrophilic, which cannot be the case of deacetylated acid products.

Current solutions and limitations. Today, standard therapeutic regimens include the surgical removal of the source of sepsis, antimicrobial therapy, optimizing oxygenation, volume resuscitation, and treatment with catecholamines. Recently, new treatment modalities have become available. Replacement of antithrombin III, continuous venous hemofiltration, application of high doses of immunoglobulins, and of low doses of hydrocortisone, have been used. Experimental aspects of treatment include the administration of C1 esterase inhibitor, pharmacological inhibition of nitric oxide (NO), plasmapheresis, the application of non-steroidal anti-inflammatory agents and of high-dose naloxone as well as manipulation of cytokines. In the last decade, the focus has shifted to the interplay between inflammation, coagulation and fibrinolysis; and in the role that the vascular endothelium plays in tying inflammation and coagulation pathways together. Xigris the only approved drug for sepsis has a narrow label, and important contraindications—in particular, the increased risk of bleeding.

The present alternative approach: Natural sophorolipids and some derivatives produced from C. bombicola have a protective effect against ongoing endotoxic shock from intra-abdominal sepsis.⁽¹¹⁻¹³⁾ Sophorolipids possess anti-viral,⁽¹⁴⁾ anti-inflammatory properties,^{(15, 16)} and decrease sepsis-related mortality in experimental sepsis when given at the time of—and well after—the septic/endotoxic insult⁽¹³⁾. Unlike some other forms of glycolipids (e.g. Lipid A) which have endotoxin effects, sophorolipids do not cause any detectable toxic effects even when administered at 100 times the therapeutic dose In addition, the production and cost of such agents is considerably lower than any sepsis therapeutic currently in development. Further in vitro investigation shows that the therapeutic effect is associated with reduced inflammatory cytokines, such as IL-1α/β, IFN-α and increased TGF-β. We postulate that sepsis-related mortality in vivo may be due to anti-inflammatory effects of sophorolipids, targeting TLR4-CD14 upstream of the inflammatory signaling cascade. Additional mechanisms involved in sepsis-related anti-inflammatory effects include reduction of nitric oxide,⁽²⁰⁾ regulation of pro-inflammatory cytokines,⁽¹⁵⁾ and modulation of cell surface adhesion molecules.⁽¹⁶⁾

Thus, the use of sophorolipids⁽¹⁹⁾ is a novel concept that offers a wide spectrum of therapeutic possibilities. They are biodegradable and have low toxicity profiles.⁽¹⁹⁾ Biological applications of sophorolipids have been reported in: cancer treatment by cytokine upregulation/macrophage activation,⁽²⁰⁻²³⁾ treatment of autoimmune disorders,⁽²⁴⁾ regulation of angiogenesis,⁽²⁵⁾ and apoptosis induction.⁽²⁶⁾

Since the natural mixture and other forms of sophorolipids obtained from bombicola do not produce a pure, reproducible molecule that can be used therapeutically under the General Manufacturing Practices (GMP) conditions required by the FDA, we have developed a non-acetylated ethyl ester sophorolipid with optimal biologic activity, trademarked as Glyco 23, for the treatment of sepsis.

Composition, Structure and Properties

Glyco 23 is a non-acetylated ethyl ester sophorolipid obtained from C bombicola by a fermentation process whereby glucose and oleic acid are added to the culture periodically over the course of 96 hours. Glyco can be dissolved in Sucrose/Ethanol or capped to a Nanoparticle for water dispersion, and injected intravenously at therapeutic doses ranging between 2 mg/Kg and 20 mg/Kg of weight.

Sophorolipids consist of a hydrophobic fatty acid tail of 16 or 18 carbon atoms and a hydrophilic carbohydrate head. Sophorose is a glucose disaccharide with an unusual β-1,2 bond and occurs as a mixture of free primary hydroxyl groups, mono acetyl or diacetyl substituents. One terminal or subterminal hydroxylated fatty acid is β-glycosidically linked to the sophorose molecule. The carboxylic end of this fatty acid is either free (acidic or open form) or internally esterified (lactonic form). The hydroxy fatty acid itself counts in general 16 or 18 carbon atoms and can have one or more unsaturated bonds: (Asner et al. 1988: Davila et at 1993). As such, the sophorolipids synthesized by C. bombicola are in fact a mixture of related molecules with differences in the fatty acid part and the lactonization and acetylation pattern. Asmer al. (1988) were the first to shed light on this structural variation. They separated the sophorolipid mixture by medium pressure liquid chromatography and thin layer chromatography, and mainly based on the lactonization and acetylation pattern, they put forward 14 components. Davila et al. (1993) separated the sophorolipid mixture by a gradient elution, high-performance liquid chromatography (HPLC) method and used an evaporative light scattering for the detection of the individual sophorolipids. They spend special attention to the analysis of the fatty acid chain and identified over 20 components.

The different structural classes cause wide variation in physicochemical properties. Lactonized sophorolipids have different biological and physicochemical properties as compared to acidic forms. Also, the presence of acetyl groups can have a profound effect on the properties of sophorlipids. Indeed, acetyl groups lower the hydrophilicity of sophorolipids and enhance their antiviral and cytokine stimulating effects (Shah et al. 2005).

### SUMMARY OF THE INVENTION

The invention is a novel class of sophorolipids compound to be used for the treatment of sepsis and septic shock induced by certain cytokines and for bacterial endotoxins, Preliminary research performed by the inventor indicates that the free acid sophorolipid displays biological activity specifically in the prevention of sepsis progression. With a primary single biologically active compound in hand future investigations may identify a defined pharmocophore within the molecule. The preparation of sophorolipids has been typified by the fermentation of various strains of Candida most particularly Candida bombicola. One method of producing sophorolipids suitable with the present invention is through microbial resting cells of Candida bombicola. Sophorolipids obtained from resting state cultures are isolated as a complex mixture of compounds.

The crude sophorolipid is decanted as a heavy oil from the culture broth, and it is washed to remove free fatty acids. The mixture of products can only be partially separated which means that it is difficult to assign biological activity to a specific pharmacophore. Secondary chemical transformation via base catalyzed hydrolysis can be used to reduce the 8 possible structural shophorolipid species to a single moiety, the 17-L-[(2′-O-b-D-glucopyranosyl-b-D-glucopyranosyl)-oxy]-cis-9-octadecenoate de-acetylated free acid.

Substantially pure Sophorolipid lactone is obtained by crystallization from ethyl acetate triturated with hexane. Glyco 23 is prepared by hydrolysis of the sophorolactone followed by esterification with sodium ethoxide which is crystallized from ethyl acetate/water. Glyco 23 can be administered to the patient intraperitoneally, or intravenously at single of multiple doses of 5 to 30 mg/kg of weight in solvent media or in capped nanoparticles.

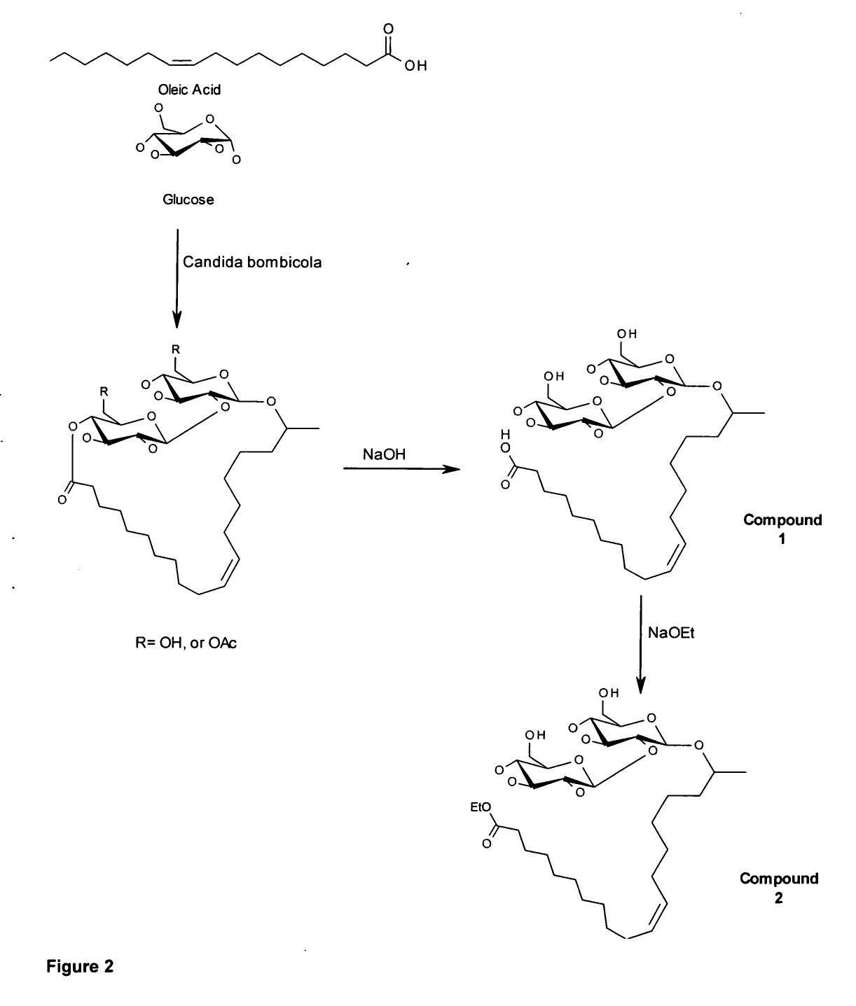

The invention consists of a process for the fed batch production of a sophorolipid composition affording a major part of at least partly acetylated acids under particularly advantageous conditions and obviating the aforementioned disadvantages. More particularly, culturing of at least one Candida bombicola strain in a culture medium incorporating a glucose carbon source and at least one nitrogen source under appropriate conditions for cultivating said strain. The said strain is then exposed to a supply of an appropriate substrate under adequate aeration, temperature and pH conditions and the following sequence is performed at least once: The synthetic scheme shown in FIG. 2 demonstrates the approach to preparing scalable multigram quantities of de-acetylated sophorolipids in free acid or ester forms. High purity sophorolipids and their derivatives may be produced efficiently by recrystallization of crude reaction products which is a significant process improvement from the described literature. Entailing labor intensive and costly silica gel column chromatography.

### BRIEF DESCRIPTION OF THE DRAWINGS

1. Mixture of compounds representing the acid form and the lactone form

2. Approach to preparing scalable multigram quantities of de-acetylated sophorolipids in free acid or ester ester forms.

3. Effect of Glyco 23 on mortality in intra-abdominal sepsis. Kaplan Meier statistics performed on survival

4. Reduction in mortality compared following administration of vehicle alone (V), ester sophorolipid derivative (e-SL), sophorolipid mixture (SL), and Lactonic derivative (L-SL)

5. Pro-inflammatory cytokine suppression: A-IL1; B-IL-8

6. Effect of Glyco 23 on cytokine production in CLP sepsis: IL-1β (A) and TGF-1β (B) in splenic lymphocytes of rats treated with natural sophorolipid mixtures using RNase Protection Assay

7. Inhibition of cytokine expression by Glyco 23 analyzed by microarray.

8. Effects of Glyco 23 on TLR pathway:

9. Expression of macrophage CD14 and TLR4 in an in vitro model system using cultured macrophages to relate Glyco 23 mechanism of action to events upstream of cytokine gene expression.

10. Inhibition of Serum IL-6 by SL in rats with polymicrobial sepsis

11. Histology samples. Data on cellular damage and protection by Glyco.

### DETAILED DESCRIPTION OF THE PREFERRED EMBODIMENTS

Description will now be given with reference to the attached FIGS. 1-11. It should be understood that these figures are exemplary in nature and in no way serve to limit the scope of the invention, which is defined by the claims appearing herein below.

The present invention is a method for producing sophorolipids and using the non-acetylated ethyl esther sophorolipid in water dispersable in nanoparticles for the treatment of sepsis and septic shock. After synthesizing the sophorolipid by fermentation of Candida bombicola in a fermentation media to form a natural mixture of lactonic sophorolipids and non-lactonic sophorolipids, the lactonic sophorolipids are isolated by crystallization and then treated in sequence first with an aqueous sodium hydroxide solution followed by esterication with and ethanolic solution of sodium ethoxide. The final ethyl-deacetylated sophorolipid is then crystallized from ethyl acetate/water mixture to provide Glyco 23.

The sophorolipid compounds disclosed herein can be delivered intravenously and intraperitoneally in water dispersable capped nanoparticles.

Dosages can be determined depending on the particular sepsis or septic shock circumstance, but generally is in the 5-30 mg per kg of body weight range as single or multiple dose several hours post inception.

The method for producing sophorolipids for prophylaxis or treatment of sepsis and septic shock in a human or animal comprises the following steps:

Fermentation and isolation of crude sophorolipid mixtures. Candida bombicola (ATCC 22214) was obtained from NRRL in Peoria Ill. and subcultured in 3 milliliters of a liquid broth composed of 100 g/L glucose, 10 g/L yeast extract and 1 g/L Urea. The starter culture was scaled into 100 milliliters of similar broth maintained at 30° C. in a sterile 500 milliliter baffled Erlenmeyer flask. The culture was used as a secondary starter culture for a 1 liter fermentation containing similar broth. One liter fermentations were performed in a New Brunswick Bio-Flo stirred tank held at 30° C., 500 RPM agitation and 0.8 liters per minute air flow. Cultivation was allowed to proceed for 48 hours following which, the fermentor was charged with 40 grams of glucose (as a sterile 50 weight percent solution) and 20 grams of oleic acid. The fermentor was again charged with 20 grams of glucose after 24 hours. A final addition of 10 grams of glucose and 10 grams of oleic acid was added after 24 hours and the ferementor was shut down 24 hours following the final substrate additions. The fermentor air and agitation was shut off and crude sophorolipids were allowed to settle to the bottom of the chamber as viscous brown oil. Biomass suspended in spent culture broth was decanted from the oil and the oil was first washed with cold deionized water to remove culture media and residual biomass. The oil was then dissolved in ethyl acetate and filtered to remove unwanted solids. The solvent in the organic mixture was then removed in vacuo and the remaining solids were washed with cold hexane to remove residual and unwanted lipids. The crude sophorolipid mixture was then dried overnight under vacuum to afford 22 grams of a grey solid. The grey solid was dissolved in a minimal amount of hot ethyl acetate and the solution was triturated with hexane to afford sophorolipid lactone mixture as puffy white crystals. The synthetic scheme shown in FIG. 2 demonstrates the approach to preparing scalable multigram quantities of de-acetylated sophorolipids in free acid or ester ester forms.

Hydrolysis and isolation of sophorolipid free acid. 10 grams of sophorolipid lactone above was dissolved in 50 milliliters of deionized water. The stirred solution was charged with 10 milliliters of 10 N NaOH at room temperature. The reaction was allowed to proceed for 2 hours during which the solution slowly dissolved and became a clear yellow solution. The reaction was cooled to 0 C in an ice bath and the pH of the reaction solution was reduced to 3 with a 1M HCL solution. The chilled aqueous solution was extracted with ethyl acetate and aqueous layer was concentrated and maintained at near 0° C. to afford a light white precipitate. Recovery and drying of the precipitate afforded 9 grams of white powder. Analysis by NMR and MS revealed the compound to be the de-acetylated free acid Compound 1.

Chemical routes to sophorolipid ethyl ester. 1 gram (1.6 mmol) of Compound 1 was added to a stirred round bottom flash containing dried ethanol under a nitrogen atmosphere. The reaction was started by addition of 20 milligrams of NaOH and the reaction was refluxed for 3 hours. The reaction was cooled in an ice bath and then neutralized by addition of acetic acid. The solvent was removed in vacuo to afford a light brown oil. The oil was dispersed in ice cold deionized water and allowed to stand overnight at 4° C. The solution formed a white precipitate which was harvested and dried under vacuum to afford 750 ml. of ethyl ester Compound 2. Non-acetylated ethyl esther sophorolipid can be dissolved in water/sucrose dispersable capped nanoparticle for the treatment of sepsis and septic shock

### EXAMPLES

### Example 1

Preparation of natural sophorolipid mixture. A single colony of Candida bonibicola ATCC 22214 cultured on GY medium in agar is cultured in 50 milliliter shake flasks in liquid GY medium at 30 C for 24 hours. This starter culture is then aseptically harvested and transferred to a 1 liter working volume stirred tank fermentor which is set to 30 C at 400 RPM and aerated at 0.8V/min. After 24 hours growth 40 grams of sterile glucose (as a 50 wt % solution) is added to the fermentor followed by 40 grams of sterile oleic acid and the culture. After 24 hours the fermentor is charged with an additional 20 grams of sterile glucose. A final 20 grams of sterile glucose is added to the fermentor after 24 hours which is followed by 24 hours of cultivation. The fermentation is stopped and the crude sophorolipid product is allowed to settle to the bottom of the reactor which is then separated by decanting the spend culture broth to afford a viscous light brown syrup. The syrup is washed with 2 times one volume of deionized water followed by extraction by 3 times 1 volume of ethyl acetate. The organic extract is then concentrated and dried under vacuum to afford a pale yellow solid. The dried solid is dissolved in minimal volume of hot ethyl acetate and then the clear solution is triturated with hexane and allowed to cool. Upon cooling sophorolipid lactone forms white fluffy crystals which are harvested by filtration.

### Example 2

Preparation of Ethyl-17-L-[(2′-O-D-glucopyranosyl-D-glucopyranosyl)-oxy]-cis-9-octadecenoate. To a round bottom flask is added 1 gram (1.6 mmol) of dried sophorolipid free acid followed by 10 milliliters of dry ethanol freshly distilled from magnesium turnings. The reaction mixture is stirred under nitrogen and charged with 20 milligrams (0.29 mmol) of sodium ethoxide powder. The reaction mixture is heated to reflux and stopped by cooling after being judged complete by the disappearance of starting material by TLC. The cooled in an ice bath and the solution is neutralized by addition of glacial acetic acid and then the solvent was removed under vacuum to afford a light yellow oil. The oil was dispersed in cold water and the ethyl ester sphorolipid was recovered as a white powder by filtration.

Since the previous version of this application, we have confirmed that sophorolipids as Glyco 23 formulation have no significant antibiotic activity at clinically relevant concentrations against a selection of standard bacterial isolates (broth microdilution method)⁽³⁵⁾.

### Example 3

Effect of Glyco 23 on mortality in intra-abdominal sepsis. Preliminary data to develop a CLP model for these studies showed that we can obtain reproducible mortality rates of 60% to 70% with a 16 gauge needle in a CLP model. The CLP model was chosen for its reproducible mortality rates and its ability to mimic fecal peritonitis.

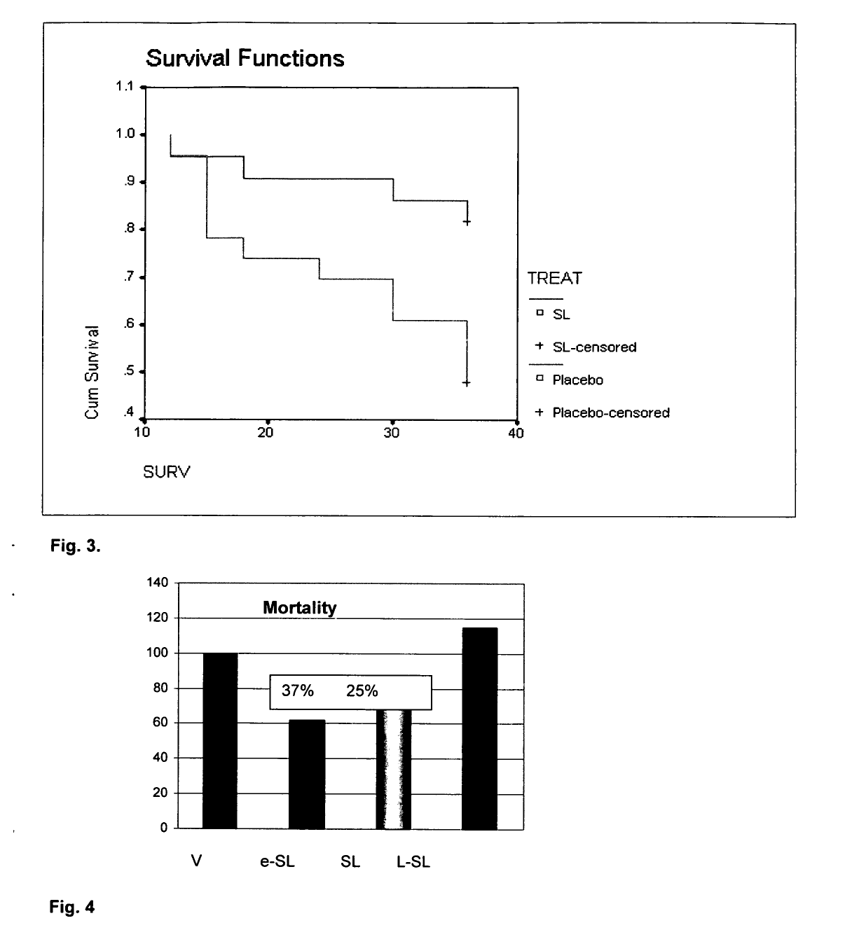

Animals were randomized into two groups: control and experimental, (n=25/group) as shown in FIG. 3 induced with septic peritonitis via CLP; and treated IV with saline or natural sophorolipid mixture (SL) (5 mg/kg). This dose is well below the LD50 (6-7 gm/kg) of naturally occurring sophorolipids in rodents.^{(40,41)}. Doses were given at the end of the surgery, and animals were followed for 36 hours. Kaplan Meier statistics were performed on survival. The 36 hr survival rate was 47.8%, and increased to 81.8% in animals treated with sophorolipid IV (P<0.05) (FIG. 3). This significant improvement in survival was achieved with a single dose of natural sophorolipid mixture given at the induction of sepsis.

In the same CLP model, reduction in mortality was compared following administration of Vehicle alone (V), ester sophorolipid derivative (e-SL), sophorolipid mixture (SL), and Lactonic derivative (L-SL) (FIG. 4) Mortality rate with Ester Ethyl Sophorolipid compared to vehicle was reduced by 37%, while natural mixture reduced mortality by 25% and Lactonic had no protective effect.

### Example 4

Survival at 3 hours and 24 hours post insult. Experimental rats were divided into seven groups (n=6/group) and induced with septic peritonitis via CLP and treated with saline or natural sophorolipid mixture, Free Acid derivative, Ethyl ester derivative and methyl ester derivative (5 mg/kg), IV. Doses were given at the end of the surgery, and animals were followed for 24 hours (Table 1)

As shown in table 1, all sophorolipid treated animals survived 24 hours compared to controls following CLP, where as the majority of the control animals died within 24 hours.

TABLE 1

| | 3 hours post insult | | | | | | |
|---|---|---|---|---|---|---|---|
| | Rat | Survival | WBC | | 24 Hs. post insult | | |
| | # | % | 10⁶ cell/MI | Blood Culture | Survival | WBC | Blood Culture |
| Saline | 6 | 84 | 6.3 ± 2.5 | E. coli, P. mirabilis, | 32 | NA | NA |
| Controls | | | | Enterococcus sp, | | | |
| | | | | E. coli, Strep vir grp, | | | |
| | | | | Staph sp Co Neg | | | |
| | | | | Enterococcus sp | | | |
| Sham | 6 | 100 | 4 ± 1.5 | Staph sp Co Neg | | | |
| Controls | | | | Strep vir grp, | | | |
| | | | | Enterococcus sp, | | | |
| Lipid A | 6 | 50 | N/A | | 32 | 5 ± 1.5 | E. coli, P. |
| | | | | | | | mirabilis, |
| | | | | | | | Enterococcus |
| | | | | | | | sp |
| Natural | 6 | 100 | — | | 89 | 4. ± 1.2 | Strep vir grp |
| Sophorolipids | | | | | | | Staphylococcus |
| | | | | | | | aureus |
| | | | | | | | Enterococcus |
| | | | | | | | sp, Staph sp |
| | | | | | | | Co Neg |
| Sohorolipid | 6 | 100 | — | | 90 | 6.4 ± 2 | Staph sp Co |
| Free Acid | | | | | | | Neg |
| derivative | | | | | | | E. coli P. mirabi |
| | | | | | | | Enterococcus, |
| | | | | | | | Staph |
| Glyco 23 | 6 | 100 | — | | 100 | 4. ± 1.6 | E. coli, P. |
| | | | | | | | vulgaris, |
| | | | | | | | Enterococcus |
| | | | | | | | Staph sp Co |
| | | | | | | | Neg, P. mirab. |
| | | | | | | | iliEnterococcus |
| Sohorolipid | 6 | 100 | — | | 87 | 4. ± 1.3 | E. coli, P. mirab |
| Methyl Ester | | | | | | | Enterococcus |
| derivative | | | | | | | |

### Example 5

Effect of delayed administration of Glyco 23 on mortality: Glyco 23 has a protective effect against endotoxic sophorolipids 2 hrs after galactosamine-EPS treatment resulted in 56% lower mortality than that observed among positive control mice (receiving only galactosamine treatment) or mice treated with Glyco 23 1 hr before or simultaneously with galactosamine-LPS treatment.

The effects of a non-acetylated esther ethyl sophorolipid trademarked as Glyco 23 was studied in a mouse model that employs galactosamine-sensitized LPS endotoxic shock induction. This model was shown to increase animal sensitivity to the lethal effects of lipopolysaccharide several thousand fold. Therefore, treatment after 2 hr can be compared to treatment after 24 hrs or later in conventional models. Glyco 23 administered to septic animals 2 hr after insult decreased endotoxin mortality by 56% (Table 2) The fact this sophorolipid demonstrated such a robust response in an accelerated animal mortality model is remarkable and provides further support of therapeutic utility.

TABLE 2
D-galactosamine model LPS LPS + Glyco Mortality (%)
Time of Injection (hrs)

| | | | |
|---|---|---|---|
| — | — | 0 | (n = 6) |
| 0 hs | — | 83 | (n = 6) |
| 1.5 hs | 0 hs | 80 | (n = 10) |
| 0 hs | 0 hs | 89 | (n = 9) |
| 0 hs | 1.5 hs | 30 | (n = 10) |

### Example 6

Effects of Glyco 23 formulation on cytokines in vitro: We have also determined the cytokine responses to the enhanced formulation of sophorolipid trademarked as Glyco 23 (ethyl ester with no acetate groups) compared to the natural mixture produced by the Gross method and derivatives.

As shown in FIG. 5, Glyco 23 and select SL isoforms decreased IL-1 and IL-8 cytokine responses when compared with the natural mixture responses. Lactonic isoform did not show suppression. Further, the mono and di-acetate ethyl ester isoforms showed different levels of suppression: 50% for IL-1, but 75% for IL-8 expression. These data suggest that select isoforms possess potent anti-inflammatory responses that may be expressed in animal models of inflammatory disease. Glyco 23 (ethyl ester with no acetate groups) showed strongest effect and was used in further studies.

### Example 7

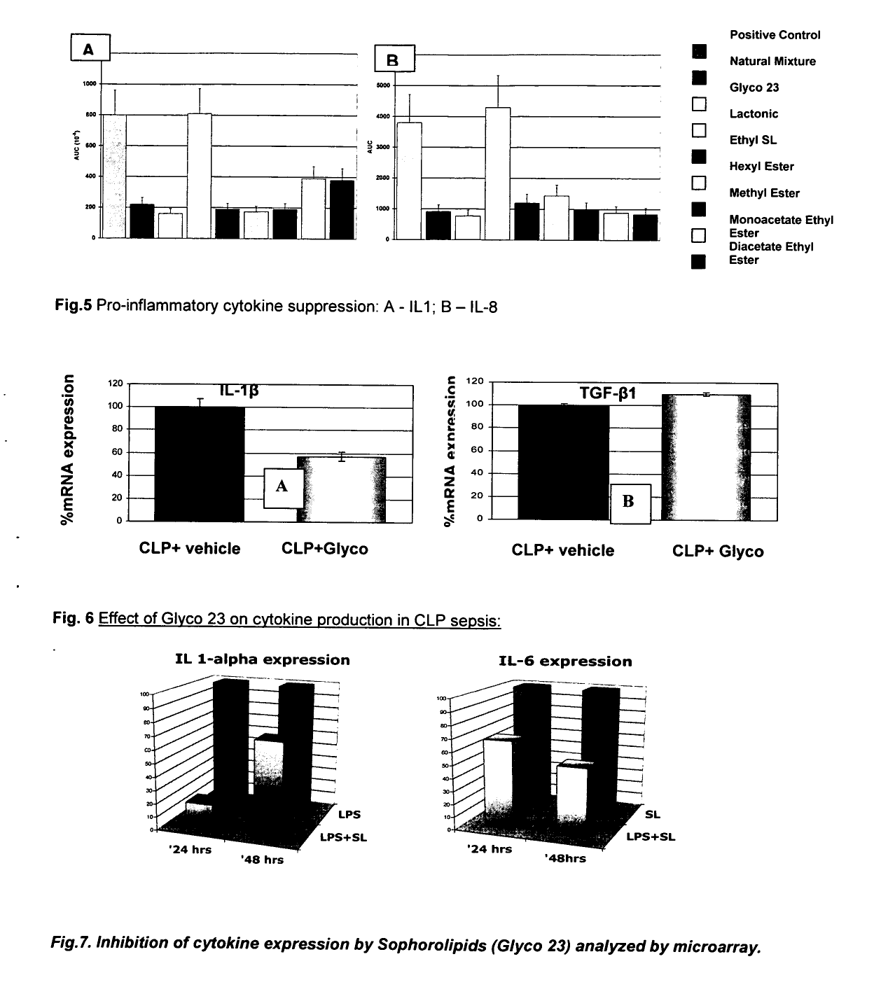

Effect of Glyco 23 on cytokine production in CLP sepsis: Using RNase Protection Assay, we demonstrated that mRNA isolated at 6 hrs from splenocytes of control CLP-septic rats expressed high levels of IL-1β (FIG. 6A). In contrast, mRNA from splenocytes of septic rats treated with Glyco 23 (5 mg/kg) had a 42.5%±4.7% (P<0.05) showed reduction in IL-1β expression (FIG. 6A). Similarly, mRNA from splenocytes of CLP-septic rats treated with saline expressed TGF-β1 (FIG. 6B), which showed an 11.7±1.5% (P<0.05) increased expression (FIG. 6B). Additional data indicated that LPS treatment alone demonstrated changes in clumping and cell viability of macrophages whereas addition of sophorolipid reversed this effect. Furthermore, sophorolipid treatment alone had no effect on cell morphology or viability (trypan blue exclusion) (not shown). Data are expressed as percent control (CLP+vehicle)+/−SEM. Treatment groups were significant (p<0.05) compared with control using student's T test. CLP=cecal ligation and puncture; SL=sophorolipid

We have also demonstrates the effect of multiple sequential (q24 hr×3 doses) IV dosing regimens of sophorolipid administration in septic rats (CLP) (79). Sophorolipid treatment showed a trend toward improved survival of rats with CLP-induced septic shock by 28% at 24 hr and 42% at 72 hr for single dose and 39% at 24 hr and 26% at 72 hr for sequential doses when compared with vehicle control (p>0.05) (79).

### Example 8

Microarray analysis of natural sophorolipid mixture mediated changes in gene expression in models of intra-abdominal sepsis and macrophages: Microarray analysis of mouse macrophages cultured with LPS+/−Glyco 23 identified groups of immunologically relevant genes whose expression was upregulated more than 5 fold by LPS (FIG. 7). The maximum level of each gene expression attained in the presence of LPS was set as 100%. Expression of these genes was suppressed in the presence of Glyco 23, demonstrating the inhibitory effect on LPS-induced cytokine production (FIG. 5). The analysis was performed using the Affymetrix GeneChip® Murine Genome Array U74Av2 probe array.

### Example 9

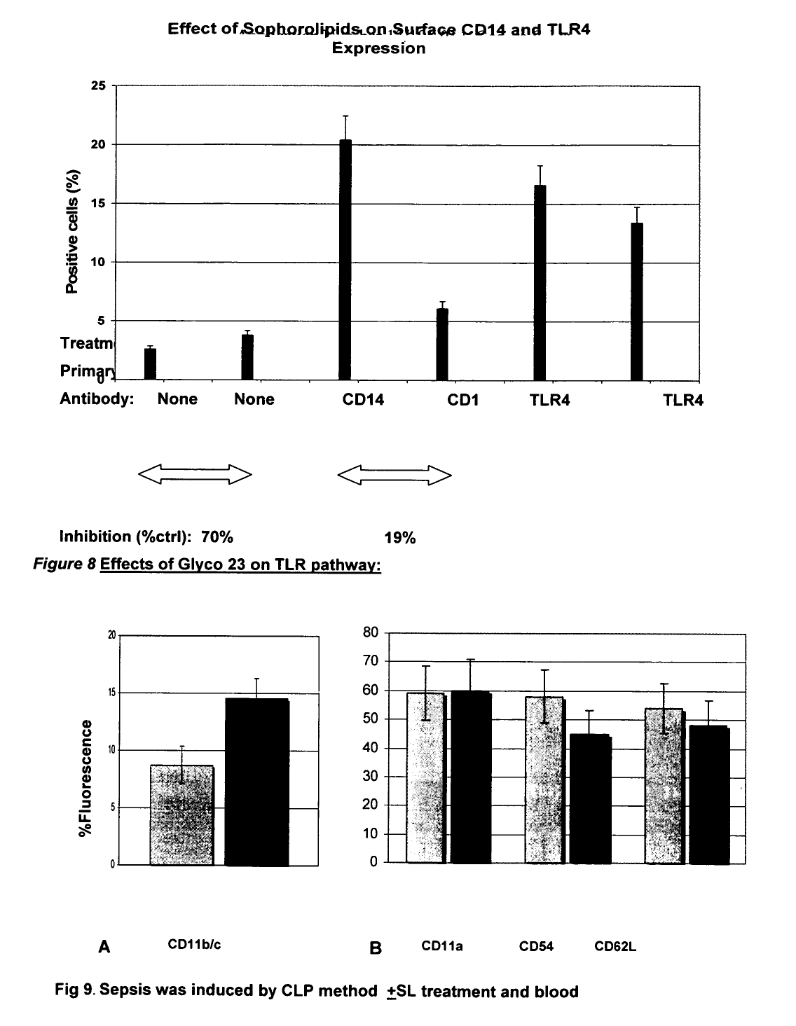

Effects of Glyco 23 on TLR pathway: We studied expression of macrophage CD14 and TLR4 in an in vitro model system using cultured macrophages to relate Glyco 23 mechanism of action to events upstream of cytokine gene expression. The results indicate that Glyco 23 interferes with surface expression of CD14 and TLR4, key components of the pro-inflammatory signal cascade in macrophages in response to bacterial endotoxins during sepsis and septic shock (FIG. 8). This observation is significant in light of very recent studies showing that CD14 inhibition using anti-CD14 antibodies in an in vivo pig model of gram-negative sepsis and endotoxemia.⁽⁴²⁾

Macrophages (RAW264.7) were incubated in the absence or presence of sophorolipids (SL, 10 μg/ml, 30 min, room temperature), washed with phosphate-buffered saline (PBS), and anti-CD 14 or anti-TLR4 antibodies (Santa Cruz Biotechnology, 10 μg/ml, 30 min, room temperature). The cells were then washed with PBS and staining was performed using ABC staining system (Santa Cruz Biotechnology), according to manufacturer's instructions. The cells were then fixed with 1% formaldehyde and examined microscopically. A total of 200 cells were scored in triplicate for each determination, and results expressed as % of total cells counted.

The results indicate that sophorolipids interfere with surface expression of CD14 and TLR4, key components of the pro-inflammatory signal cascade in macrophages in response to bacterial endotoxins during sepsis and septic shock. This observation is significant in light of very recent studies showing that CD14 inhibition using anti-CD14 antibodies in an in vivo pig model of gram-negative sepsis and endotoxemia (Thorgersen E B, Hellerud B C, Nielsen E W, Barratt-Due A, Fure H, Lindstad J K, Pharo A, Fosse E, Tonnessen T I, Johansen H T, Castellheim A, Mollnes T E. CD14 inhibition efficiently attenuates early inflammatory and hemostatic responses in Escherichia coli sepsis in pigs. FASEB J 2010; 24:712-722.

### Example 10

Effect of Glyco 23 on adhesion molecules. We have previously demonstrated that sophorolipids decreased sepsis related mortality in vivo in a rat model of peritonitis and in vitro by analysis of cytokine production. In order to better understand possible mechanisms of sophorolipid action, we investigated changes in cell surface expression profiles of helper/cytotoxic T cells (CD4, CD8), and adhesion molecules including ICAM (CD54), L-selectin (CD62L) and integrins (CD11a, CD11b/c) on blood leukocytes obtained from sophorolipid treated septic rats, compared with untreated and sham (laparotomy) controls (FIG. 9). Intra-abdominal sepsis was induced in rats via cecal ligation and puncture (CLP). Sophorolipids (SL) (5 mg/kg) or vehicle alone were injected intravenously (IV) via tail vein at the end of the operation

Sophorolipid treated rats showed a 67% increase in lymphocyte CD11b/c expression when compared with untreated controls (15% vs. 9%, respectively, p<0.05) (FIG. 9A). Sophorolipid treatment also demonstrated a trend toward decreased lymphocyte CD54 and CD62L expression when compared with untreated controls (59% and 45%, respectively; 55% and 47%, respectively, p>0.05), and lymphocyte CD11a expression was similar in both groups (FIG. 9B). CD4+ and CD8+ cells were significantly reduced in both CLP groups (±sophorolipid treatment) when compared with sham group (7%±1% and 13%, respectively; 8%±2% and 39%, respectively, p<0.05) (data not shown).

### Example 11

Dosage To determine the optimum dose that can be administered to the rats, 3 different doses of Glyco 23, 6 mg/kg, 12 mg/kg and 24 mg/kg of rat body weight (in 50% ethanol-PBS) was administered using tail vein IV to 9 rats following the Cecal Ligation and Puncture (CLP). Control rats (n=3) were injected with 0.5 ml of PBS. All rats that received 24 mg/kg dose died within 2 hours indicating that this dose might be lethal due to the ethanol concentration in the media. Rats that received 8 mg/kg and 12 mg/kg doses survived after 24 hours. Blood was obtained before they were sacrificed the next day.

### In Vitro Experiments: Mononuclear Cell Response to Sophorolipids in Rats:

Peripheral blood mononuclear cells (PBMC) were obtained from nine rats 24 hrs after intravenous injection of sophorolipids:

1. Control (PBS) (n=3)

2. Sophorolipid (6 mg/kg, IV) (n=3)

3. Sophorolipid (12 mg/kg, IV) (n=3)

Blood (2 ml/animal) was collected with anticoagulant (EDTA, purple top tubes) and PBMC isolated by Ficoll-Hypaque discontinuous gradient centrifugation. PBMC from individual animals were placed in 24-well tissue culture wells in 1 ml of minimal essential medium supplemented with 10% fetal calf serum, 2 mM glutamine and antibiotics (penicillin, 500 U/ml, streptomycin, 500 μg/ml, bacitracin, 25 μg/ml) (complete MEM).

Cultures were incubated for 24 hrs at 37° C. in a humidified atmosphere containing 5% CO₂. Non-adherent cells were removed by pipetting; 1 ml of fresh complete MEM was added to each culture with the remaining adherent cells (monocytes), and these cultures were examined using an inverted microscope. A minimum of 4 microscopic fields were examined for each culture, and the number and appearance of adherent cells/field determined. The data are summarized in the table below.

| GROUP | Cells/field ± SEM | Estimated cell diameter (μm) |
|---|---|---|
| 1 | 36 ± 4 | 18 |
| 2 | 68 ± 5 | 32 |
| 3 | 69 ± 3 | 33 |

Monocyte appearance, numbers, and size suggest that at 6 mg/kg, sophorolipids may be sufficient to cause monocyte activation. Culture supernatants will be collected at 5 days and the amounts of characteristic monocyte/macrophage activation products (nitric oxide, TNF-α) will be determined using the modified Griess reaction and ELISA, respectively.

We determined that 12 mg/kg of rat body weight was the optimum dose and was used for the subsequent experiments.

### Example 12

### Cytokine Response Inhibition of Serum IL-6

Serum samples were obtained from rats (N=5 animals/group) 3 hrs. after (CLP) to establish polymicrobial sepsis, and intravenous injection of saline (control), sophorolipids-natural mixture (SL) or Glyco 23. Serum concentrations of Interleukin-6 (IL-6) were determined using commercial ELISA tests according to manufacturer's instructions. Results were evaluated for statistical significance using ANOVA.

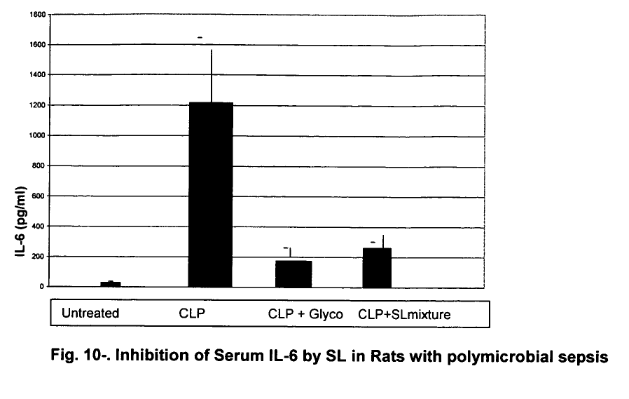

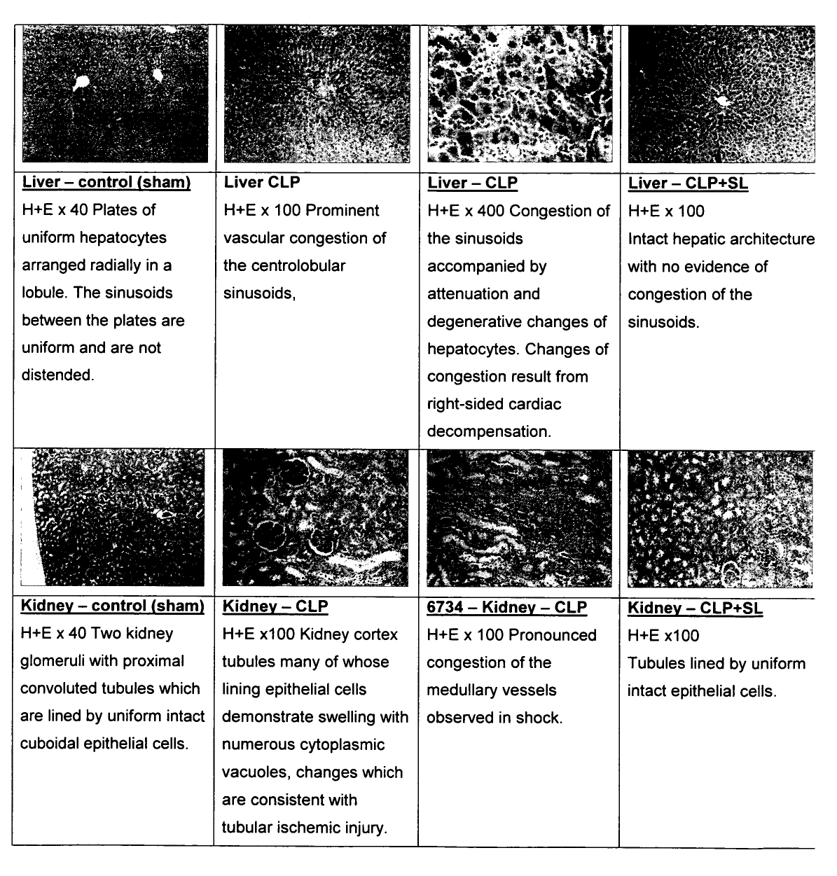

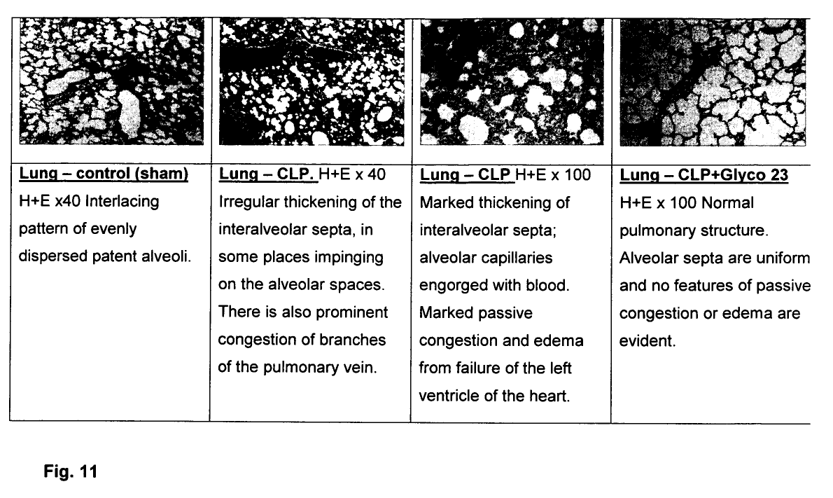

It was observed that serum IL-6 increased dramatically (more than 40-fold) at 3 hrs after CLP procedure, and that administration of sophorolipids was accompanied by a profound reduction (12-fold) of serum IL-6 concentrations; although they did not reach the low levels observed in sham-treated animals. No significant difference was observed between the inhibitory effects of natural mixture and free acid form of sophorolipids (FIG. 10).

### Example 13

Histology Tissue samples were taken from the lung, liver and kidney at 24 Hs. post insult. Tissue was fixed in formaline and embedded in paraffin and sections cut at 5 micron. Treated animals were injected with the sophorolipid mixture. Data on cellular damage and protection by Gluco is described in FIG. 11

Having described certain embodiments of the invention, it should be understood that the invention is not limited to the above description or the attached exemplary drawings. Rather, the scope of the invention is defined by the claims appearing hereinbelow and any equivalents thereof as would be appreciated by one of ordinary skill in the art.

What is claimed is:

1. A process for the fed batch production of a sophorolipid composition for the treatment of sepsis comprising the steps of:
culturing Candida bombicola strain in a culture medium incorporating a sugar and a nitrogen source under effective conditions for producing said strain and, thereafter, exposing said cultured strain in a reaction zone to a supply of a substrate under adequate aeration, temperature and pH conditions, said substance consisting essentially of at least one animal oil, at least one vegetable oil, and/or at least one ester of said oil, said oils and said ester incorporating an aliphatic c linear chain with 10 to 24 carbon atoms, and wherein the following sequence is performed at least once:
(a) continuously supplying the substrate to the strain culture at a flow rate in the reaction zone between 1 and 4 grams per hour and per liter of initial volume and for a supply time such that the residual concentration of said substrate in the reaction zone is maintained at a value at the most equal to 18 grams per liter of initial reaction volume during said supply time and producing the sophorolipids while said reaction zone is essentially free of sugar during at least part of said supply time, and
(b) recovering the resultant sophorolipid composition having a non-acetylated acid form.

2. A process according to claim 1, wherein the sophorolipid composition recovery stage comprises the separation of the strain from the fermentation liquid containing the sophorolipid composition, neutralization at a pH close to neutrality of the liquid and the elimination of the water by heating and under reduced pressure.

3. A process according to claim 1, wherein the sophorolipid composition recovery stage comprises the separation of the strain from the fermentation liquid containing the sophorolipid composition and the elimination of the water under reduced pressure.

4. A process according to claim 1, wherein the substrate supply is stopped when the total quantity of injected substrate reaches at the most 280 g/l of initial reaction volume.

5. A process according to claim 1, wherein the sophorolipids are produced at a temperature of 15° C. to 35° C., a pH of 2.5 to 8, and wherein the reaction zone is aerated at a rate of 0.2 to 2 wm. under a pressure of 1 to 5 bar.

6. A process according to claim 1, wherein the substrate consists of at least one colsa, sunflower, palm and/or soy oil and at least one ester of said oils.

7. A process according to claim 1, wherein the substrate flow rate in the reaction zone is between 1.0 and 3.0 g/minute of initial reaction volume.

8. A process according to claim 1, wherein the strain comes from a culture produced ex-situ.

9. A process according to claim 1, wherein the strain contained in the culture medium is exposed to the substrate supply.

10. A process according to claim 1, wherein the reaction zone contains at the start of culturing a substrate concentration of 5 to 40 g/l of initial reaction volume and said strain is continuously supplied with substrate when the initial substrate concentration is 1 to 5 g/l

11. A process according to claim 1, wherein the quantity of cells used based on the reaction volume is 1 to 100 g of dry weight per liter.

12. A process according to claim 1, wherein, prior to the culturing stage, at least one preculturing stage of the strain is performed under appropriate conditions incorporating at least one carbohydrate, at least one saturated or unsaturated fatty acid ester with 10 to 24 carbon atoms, and at least one saturated or unsaturated aliphatic hydrocarbon with 10 to 20 carbon atoms.

13. A process according to claim 12, wherein the preculture medium comprises as the carbon source a carbohydrate and at least one other carbon source chosen from the group consisting of esters, hydrocarbons, alcohols and acids. at least one aliphatic alcohol with 10 to 20 carbon atoms, at least one aliphatic acid with 10 to 20 carbon atoms or mixtures thereof, the carbohydrate proportion being at the most equal to 20% and preferably between 2 and 12% based on the preculture medium and the weight proportion of ester, hydrocarbon, alcohol and/or acid is below 6.5%, preferably between 0.1 and 0.3% based on the preculture medium and the culture medium is seeded with the preculture medium.

14. A process according to claim 1, wherein the sophorolipid comprises at least 60% of the acetylated acid form.

15. A process according to claim 1, wherein the sophorolipid comprises at least 70-90% of the acetylated acid form.

16. A process according to claim 1, wherein the sophorolipids are produced while said reaction zone is essentially free of sugar during most of the supply time.

17. A process according to claim 1, wherein the sugar is glucose.

18. A method of treatment of sepsis or septic shock comprising the steps of administering a therapeutically effective amount of a composition comprising a non-acetylated Ethyl ester sophorolipid.

19. A method according to claim 18, wherein the sophorolipid can be dispersed and delivered in solution.

20. A method according to claim 18, wherein the sophorolipid is administered in a dose of between about 2 mg to 15 mg of the mixture per kilogram.

21. A method according to claim 18, wherein the composition has the formula Ethyl-17-L-[(2′-O-b-D-glucopyranosyl-b-D-glucopyranosyl)-oxy]-cis-9-octadecenoate.
